(19) United States
(12) Patent Application Publication
(10) Pub. No.: US 2011/0319972 A1
(43) Pub. Date: Dec. 29, 2011

(54) VENOUS WARMING DEVICE

(76) Inventors: Wayne Skloss, San Antonio, TX (US); Timothy Hite, San Antonio, TX (US); Cecil Brown, San Antonio, TX (US)

(21) Appl. No.: 13/254,098

(22) PCT Filed: Mar. 4, 2010
(86) PCT No.: PCT/US10/26166

§ 371 Date: Aug. 31, 2011

Related U.S. Application Data
(60) Provisional application No. 61/157,402, filed on Mar. 4, 2009.

(51) Int. Cl. A61F 7/08

(52) U.S. Cl. 607/114

(57) ABSTRACT

An apparatus for increasing venous blood flow to an extremity of a patient is disclosed. The apparatus includes a forced heated air unit, and a detachable transparent housing having an opening through which the extremity of the patient may be introduced in to the detachable transparent housing.

This application claims priority to U.S. Provisional Patent Application Ser. No. 61/157,402, filed on Mar. 4, 2009, entitled VENOUS WARMING DEVICE, the entire content of which is hereby incorporated by reference.

### FIELD OF THE INVENTION

The present invention generally relates to an apparatus and method for increasing the venous blood flow of a living body. More particularly, the present invention is directed to an apparatus and method for use in increasing the venous blood flow of a human hand or foot, so that venepuncture may occur.

### BACKGROUND

It has been known for some time that increased blood flow to a particular portion of a body will occur upon heating of that particular portion. Low blood flow to a given portion of a body may interfere with medical treatment. For example, if a patient's veins are collapsed, it is very difficult to perform venepuncture to extract blood for testing, or to insert intravenous fluids. Therefore, it would be desirable to develop an apparatus and method to increase the venous blood flow of a patient for improved venepuncture.

A primitive system of increasing blood flow to a human hand is known in the art. Specifically, a closed wooden box in which a heating lamp was placed has been known to be used. A method of using this system included placing a patient's hand in the box and supplying heat by means of the heating lamp. However, several problems developed with the use of this system. First, since the box was closed, it was impossible to view the patient's hand while it was being heated. Therefore, an attendant was required to continually remove and inspect the hand to determine whether the patient was in a condition for venepuncture. Second, there were also many instances of patients being burned by the heating lamp of the apparatus. The primary use of the invention was with diabetic patients, who generally have poor circulation. Thus, diabetic patients could not sense that their hands were being burned by the heating lamp. The prior solution to this problem was to wrap a patient's hand in a towel to prevent burning. However, this did not solve the problem, as the towel-wrapped hand could still contact the heating lamp, causing it to be burned.

What is needed in the field is a sterile, controlled, safe, and convenient apparatus for warming an extremity or portion of a patient or animal boy. It would be advantageous to allow viewing of the patient's extremity without the need for removal, and also to prevent the possibilities of burns through the use of a forced heated air heating unit. In addition, the present invention provides a detachable transparent housing for receiving the patient's extremity, which can be removed and sterilized without disassembling the entire apparatus.

### SUMMARY

It is therefore an advantage of the present invention to provide an apparatus to increase blood flow in which the patient's extremity is capable of being viewed while inside the apparatus. It is another advantage of the present invention to provide an apparatus in which the heating element is isolated from the patient's extremity, therefore preventing the patient from accidently contacting the heating element. It is a further advantage of the present invention to provide an apparatus with further safety features to limit the heating element from operating at an undesirably hot temperature.

An exemplary apparatus constructed according to the present invention includes a detachable transparent housing having an interior and exterior, an opening in the detachable transparent housing which permits introduction of a hand into the detachable transparent housing, and a forced heated air unit comprising a heating element and a fan. Generally, it is desired that the detachable transparent housing have at least one substantially transparent portion, and be removably attachable to the forced heated air unit such that heated air may be accelerated from the forced heated air unit to the detachable transparent housing. The detachable transparent housing may be many different shapes and sizes and may be constructed of many different materials, and is removable for sterilization. The detachable transparent housing may be made of any material capable of withstanding the operating temperature of the apparatus, for example, wood, glass, polycarbonate, insulated steel, lexan, polyurethane, polystyrene, TEFLON, acrylic, plexiglass or the like. The heating element may also be many different shapes and sizes and may be constructed of many different materials. For example, the heating element may be a ceramic heater, a coil heater, heating wire, heating tape, transparent heater, or the like. Further, the heating element may be located at various positions within the forced heated air unit, depending on the heating element used. The control for the heating element may take many different forms. The heating control may be, for example, a microcontroller, a thermostat, rheostat or the like.

The apparatus may further include several safety features, for example, a safety switch capable of shutting off the heating element if the temperature in the detachable transparent housing rises above a predetermined temperature. The safety switch may be, for example, a thermal switch, an electromechanical switch such as a relay, thermal snap switch, or the like. Further, the apparatus of the present invention may include a fan assembly disposed in the detachable transparent housing to uniformly distribute heat throughout the apparatus.

The present invention also contemplates a method of using the apparatus which includes applying the desired temperature to the apparatus, placing the patient's extremity in the apparatus, viewing the extremity, removing the extremity when it has attained the desired blood flow, and performing venepuncture. The present invention may also be used in conjunction with dialysis treatment. In such a method, the apparatus may be used as a shunt to take over the function of the liver during dialysis treatment.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a hardware assembly diagram of an apparatus constructed according to the present invention;

FIG. 2 shows a flow chart diagram of a method according to the present invention;

FIG. 3 shows a complete apparatus for increasing venous blood flow in a preferred embodiment of the current invention. The apparatus includes a detachable transparent housing 5, a front portion 10, a ring 22, a flexible iris 15, a forced air unit 20 comprising heated air vents 30 and cool air vents 32, a heating element 25, and an on/off switch 27;

FIG. 4 shows a detachable transparent housing 5 in a preferred embodiment of the current invention. The detachable transparent housing 5 includes a front portion 10, a ring 22, and a flexible iris 15;

FIG. 5 shows a view from below of a forced heated air unit 20 of an apparatus in a preferred embodiment of the current invention. The forced heated air unit 20 includes a fan 200, a cool air vent 205, a heating tape 210, 220, a heated air vent 230, a heat safety cut off switch 240, an on/off switch 250, and a heat controller 255; and

FIG. 6 shows a forced heated air unit of an apparatus in a preferred embodiment of the current invention. The forced heated air unit 20 includes a cool air vent 205 and a heated air vent 230.

FIG. 7 shows a complete apparatus for increasing venous blood flow in a preferred embodiment of the current invention. The apparatus includes a detachable transparent housing 5, a front portion 10, a ring 22, a flexible iris 15, a forced air unit 20, a heating element 25, and clips 35.

FIG. 8 shows a complete apparatus for increasing venous blood flow in a preferred embodiment of the current invention. The apparatus includes a detachable transparent housing 5, a front portion 10, a ring 22, a flexible iris 15, a power unit 40, a holding unit 45, a detachable plastic tray 50, clips 35.

FIG. 9 shows a complete apparatus for increasing venous blood flow in a preferred embodiment of the current invention. The apparatus includes a detachable transparent housing 5, a front portion 10, a ring 22, a flexible iris 15, a forced air unit 20, a heating element 25, a power unit 40, a holding unit 45, a detachable plastic tray 50, clips 35, heated air vents 30, and cool air vents 32.

### DETAILED DESCRIPTION OF PREFERRED EMBODIMENTS

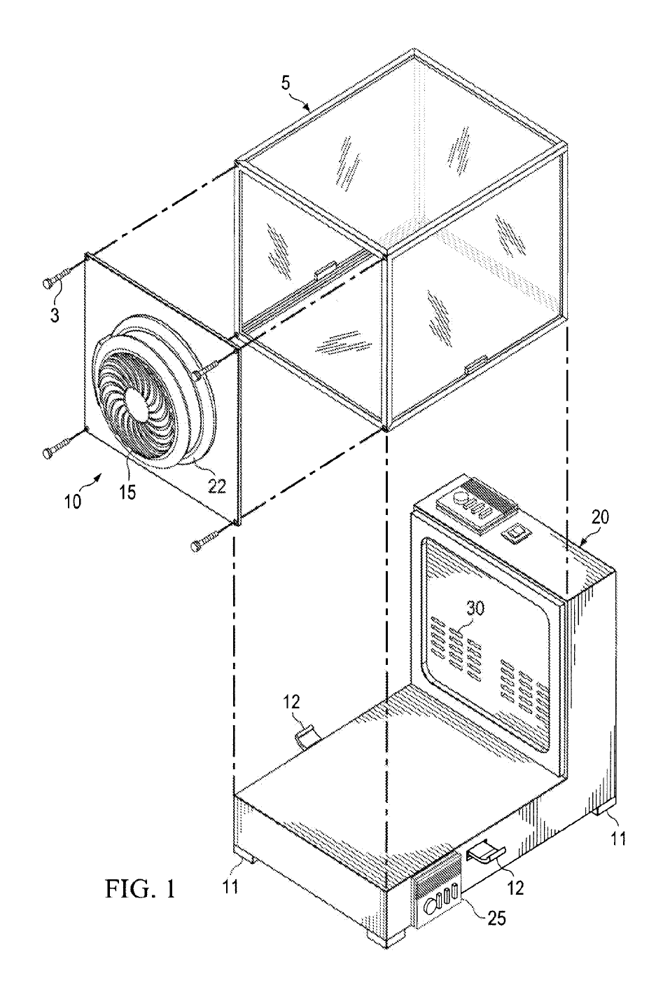

FIG. 1 shows an isometric view of an apparatus according to the present invention. The apparatus includes a detachable transparent housing 5, a front portion 10, a flexible iris 15, a forced air unit 20, and a heating element 25.

The detachable transparent housing 5 may be many different shapes and sizes and may be constructed of many different materials. The detachable transparent housing 5 may be made of any material which allows observation of an object within the housing from the outside, and which is capable of withstanding the operating temperature of the apparatus, for example, glass, polycarbonate, lexan, polyurethane, polystyrene, TEFLON, acrylic, plexiglass or the like. The detachable transparent housing is capable of being separated from the front portion 10, and the forced air unit 20, for example to be cleaned or sterilized. In an exemplary embodiment, the detachable transparent housing 5 may be rectangular in shape and may be constructed of a clear rigid material, for example, polycarbonate. A material should be selected which is capable of withstanding a temperature somewhat greater than the intended operating range of the apparatus, which is approximately between ambient temperature and 66° C., preferably between approximately 55° C. and 61° C. In an exemplary embodiment, the detachable transparent housing 5 may be constructed of five rectangular portions of polycarbonate, which may be interconnected by means of, for example, fasteners such as screws or bolts, to form a rectangular module having an open end. Alternately, the rectangular portions may be interconnected by a sealing compound, such as glue, rubber cement, or the like. The detachable transparent housing 5 may preferably be removed for sterilization without disassembling the entire apparatus.

The apparatus of the present invention may be constructed in different sizes to accommodate patients' extremities, for example, hands or feet. In an exemplary embodiment, the detachable transparent housing 5 may be approximately 11 inches high, 10 inches wide, and 12 inches deep. The present invention may be different sizes and be adapted to fit various sizes of hands, feet, or other extremities.

The open end of the detachable transparent housing 5 may be closed with a front portion 10, which may be, for example, another portion of polycarbonate. This front portion 10 may be connected to the rectangular module in any of a number of ways known to those of skill, for example, by knurl knobs 3. Such knobs provide for a quick method of removing the front end for cleaning the interior of the detachable transparent housing 5. Screws, bolts, glue or other fasteners may be used in place of knurl knobs 3. Other methods of connecting the front portion 10 to the detachable transparent housing 5 may include, for example, a hinged door which is capable of locking closed. The front portion 10 further includes an opening through which a patient's extremity may be inserted. A ring 22, capable of rotating, and which may be made of a resilient material, such as TEFLON, polycarbonate, polyvinyl-chloride, polyurethane, polystyrene or the like, is placed within the opening. A gasket may be placed around the ring to properly seal the ring 22 to the front portion 10. Further, around this ring, a flexible iris 15, such as is used in a baby incubator, a rubber insert scored with openings, or the like, is attached. In a preferred embodiment, incubator iris model Air Shields, P/N 6812074, may be used. Thus, by rotating the ring 22, the flexible iris 15 is opened and a patient's extremity may be placed within the apparatus. The ring 22 may then be rotated to close the flexible iris 15, thereby creating a tight fit around the patient's extremity, substantially sealing the detachable transparent housing 5 to maintain the heat level within the housing at the desired temperature.

The detachable transparent housing 5 may be removably attached to a forced heated air unit 20, and may be attached via fasteners 12 such as buckles, clips, clasps, screws, hook and loop closures such as Velcro®, zippers, snaps, or the like. The forced heated air unit 20 may comprise a casing, an interior, a heating element, an inlet and an outlet. The forced heated air unit 20 may further comprise a blower or fan capable of accelerating air through the inlet, where the air is exposed to the heating element and forced out the outlet. The outlet of the forced heated air unit 20 may further comprise heated air vents 30. The heating element may be many different shapes and sizes and may be constructed of many different materials. For example, the heating element may be a ceramic heater, a coil heater, heating wire, heating tape, transparent heater or the like, contained within the forced heated air unit 20.

The detachable transparent housing 5 may be attached to the top of the forced heated air unit 20, preferably such that the heated air vents 30 are in fluid communication with the interior of the detachable transparent housing 5. However, the detachable transparent housing 5 need not be on top of the forced heated air unit 20, and may be on the bottom or side, or around the perimeter of the forced heated air unit 20.

The forced heated air unit 20 may be raised by means of four spacers 11, which provide for heat to be released from the bottom of the forced heated air unit 20. It is to be noted, however, that the apparatus of the present invention may be used with the front portion 10 on a side, on top, or on bottom. In these various configurations, the spacers 11 would be placed to orient the apparatus as desired. The present discussion however is directed to use of the apparatus with the front end 7 parallel to a vertical axis.

The forced heated air unit 20 may also include a heating control 25. The heating control 25 may be, for example, a microcontroller, a thermostat, rheostat or the like. In an exemplary embodiment, heating control 25 will be a microcontroller that may include for example, a temperature controller and a solid state relay. The heating control 25 may be, for example, a miniature microprocessor temperature controller, such as Omega, CN9121A. The controller is preferably selected to operate within a 0.1° C. resolution up to a temperature of 200° C. A user can control the temperature of the apparatus by means of the main display of the heating control 25, which may include, for example, a plurality of user function keys.

In the illustrative embodiment, operation of the apparatus is controlled through a main control panel on the front face of heating element control 25. Power is turned on to the apparatus, and temperature is set to the desired temperature. After the desired temperature is set, the unit is preferably allowed to run for approximately 30 minutes to stabilize at the desired temperature. The desired temperature is in the range of 55-61° C., and more preferably at 58° C. However, individual patients may require slightly more or less heat to achieve the benefits of this invention.

As a further safety precaution, it is recommended that a user place a reference thermometer, preferably a NIST certified thermometer, in the center of the unit during stabilization. The temperature of the reference thermometer will increase higher than the temperature shown on the heating control 25 main control panel, and possibly read as much as 10° C. to 12° C. degrees higher before stabilizing to the set operating temperature.

After the apparatus has reached the desired temperature after the stabilization period, the patient procedure may begin. Ring 22 around the iris enclosure is rotated to open flexible iris 15. Next, a patient's extremity is introduced into the detachable transparent housing 5. Preferably this extremity will be a hand, but the present apparatus may also be used with a patient's foot or forearm or leg. After introduction of the hand, ring 22 is rotated to close, thereby maintaining a relatively tight seal around the patient's hand. The patient's hand can be viewed from above or aside the apparatus. When it appears that the desired blood flow has been achieved, and the patient's veins are nearer the surface, the hand may be removed from the apparatus. Typically, this may occur on the order of approximately 10-15 seconds after introduction of the hand to the apparatus. After the hand is removed, venepuncture may be performed on the patient.

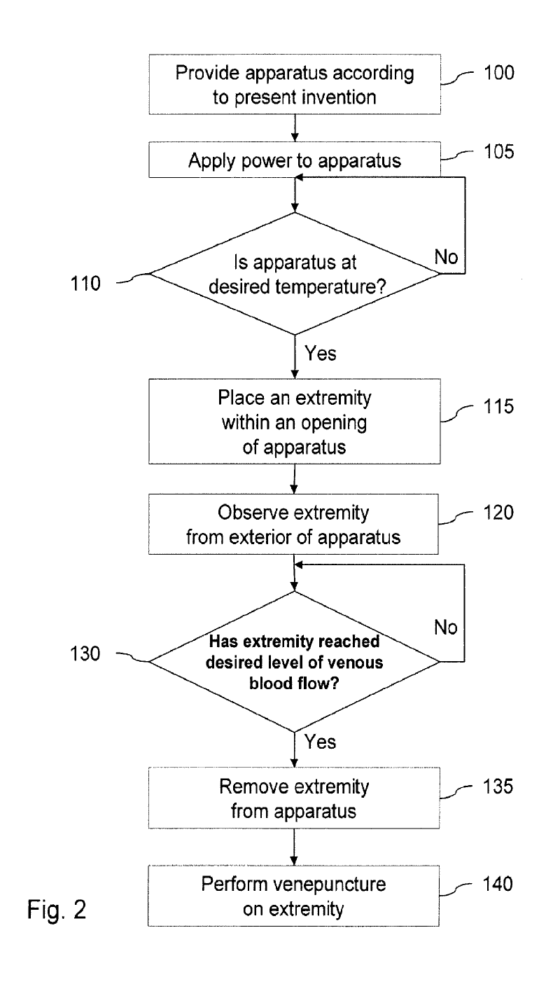

FIG. 2 shows a flow chart of a method according to the present invention. In step 100, an apparatus according to the present invention is provided. In step 105, power is applied to the apparatus, thereby placing the apparatus in an operating condition. The apparatus is set for a desired temperature, which in an exemplary embodiment may be between 55°-61° C. In step 110, the temperature of the apparatus is examined to determine whether the desired temperature has been attained. If the desired temperature has not been attained, step 110 is repeated until the desired temperature has been attained. Once the desired temperature has been attained, step 115 comprises placing a patient's extremity within an opening of the apparatus. In step 120, the extremity is observed from the exterior of the apparatus. In step 130, an operator may determine whether the extremity has reached the desired level of venous blood flow. If it has not, step 130 is repeated until the desired level is reached. If the patient's extremity has reached the desired level of venous blood flow, the extremity is removed from the apparatus in step 135. A further step of 140 may include performing venepuncture on the extremity.

In an alternate method of use after venepuncture, a patient's hand may be placed back in the apparatus. This method may be performed when the system is used in conjunction with treating liver and pancreatic disorders.

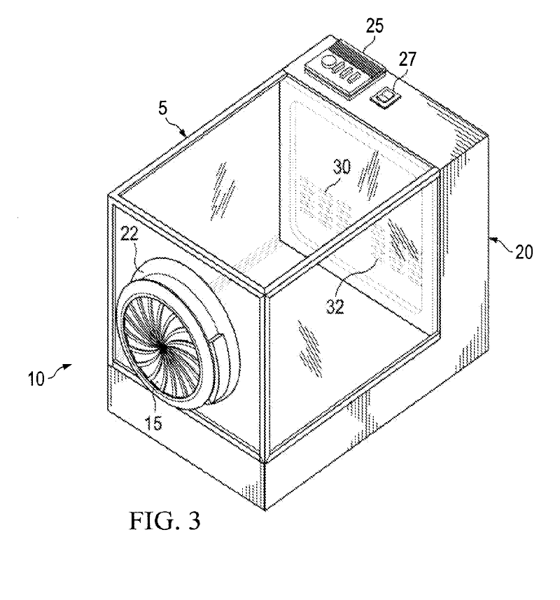

FIG. 3 shows an apparatus according to the present invention. The apparatus includes a detachable transparent housing 5, a front portion 10, a flexible iris 15, a forced air unit 20, and a heating element 25.

In this embodiment, the detachable transparent housing 5 is fitted with a front portion 10. The front portion 10 comprises a ring 22, and a flexible iris 15, which is capable of receiving a patient's extremity into the interior of the detachable transparent housing 5. The detachable transparent housing is removably fastened to a forced heated air unit 20. The forced heated air unit 20 includes cool air vents 32, heated air vents 30, a heating control 25, and an on/off switch 27. In a preferred embodiment, air will be drawn into the forced heated air unit 20 through the cool air vents 32, warmed inside the forced heated air unit, and circulated out through the heated air vents 30 into the detachable transparent housing 5. In a preferred embodiment, the air within the detachable transparent housing 5 can be adjusted to a temperature of less than approximately 66° C., preferably between approximately 55° and approximately 61° C. In another preferred embodiment, the air within the detachable transparent housing 5 can be adjusted to a temperature which results in a skin temperature of approximately 39° C. and approximately 42° C.

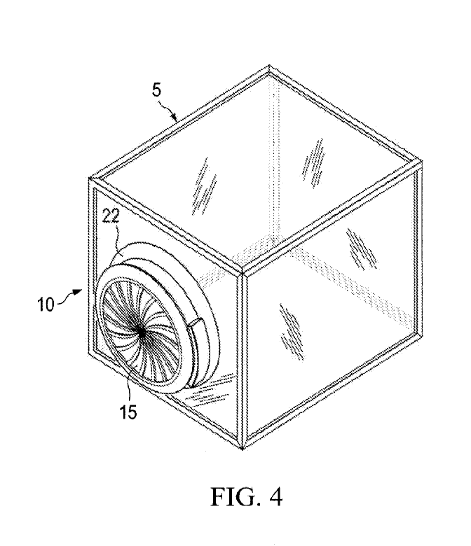

FIG. 4 shows a detachable transparent housing 5 according to the current invention. The detachable transparent housing 5 includes a ring 22, a flexible iris 15, and a front portion 10. In this embodiment, the detachable transparent housing may be removed to be cleaned or sterilized.

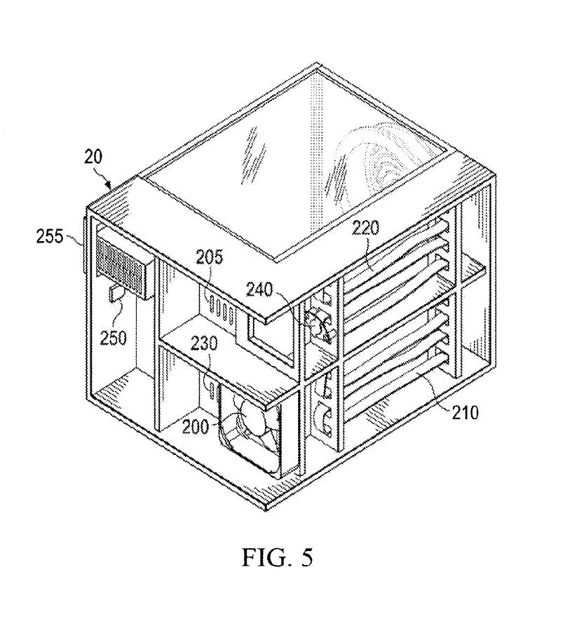

FIG. 5 shows a bottom view of a forced heated air unit 20 in accordance with the present invention, wherein wiring to the electronic components has been omitted to show detail. The forced heated air unit includes a fan 200, a cool air vent 205, a heating tape 210, 220, a heated air vent 230, a heat safety cut off switch 240, an on/off switch 250, and a heat controller 255. In a preferred embodiment of the invention, the heat controller 255 starts the heating tape 210, 220, and the fan 200. Air flows through the cool air vent 205 into the forced heated air unit 20, and returns out through the heated air vent 230 to the detachable transparent housing 5. The heat safety cut off switch 240 may automatically shut the system off if the heat controller 255 fails to shut the system off and the temperature rises above a certain level. In a preferred embodiment, the heat safety cut off switch will shut the system off if the temperature rises above approximately 66° C.

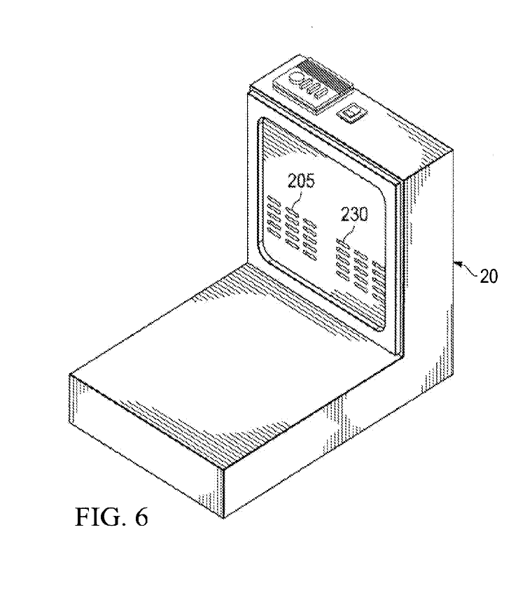

FIG. 6 shows a top view of a forced heated air unit 20 in accordance with the present invention. The forced heated air unit 20 includes a cool air vent 205 and a heated air vent 230. In a preferred embodiment, air will be drawn into the forced heated air unit 20 through the cool air vents 32, warmed inside the forced heated air unit, and circulated out through the heated air vents 30. The detachable transparent housing (not shown) may be removably attached to the forced heated air unit 20.

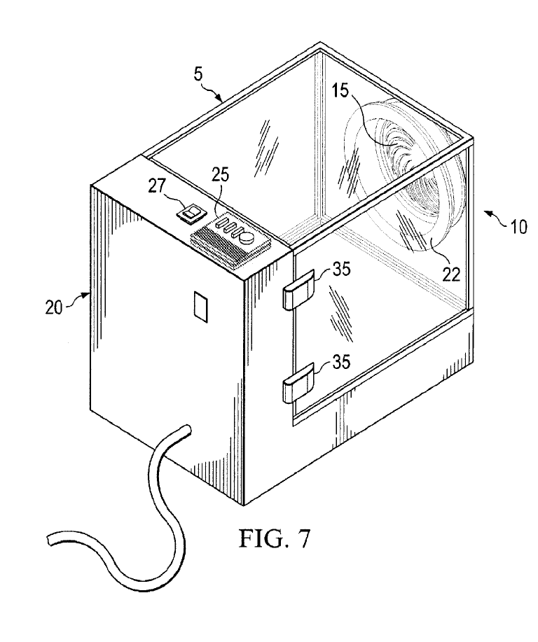

FIG. 7 shows a complete apparatus for increasing venous blood flow in a preferred embodiment of the current invention. In this embodiment, clips 35 provide a means for removably securing a power unit 40 to a holding unit 45. This allows for removal of the holding unit, which comprises a housing 5, a front portion 10, a ring 22, a flexible iris 15, and a detachable plastic tray 50, from the power unit 40, for purposes of cleaning or sterilizing the holding unit.

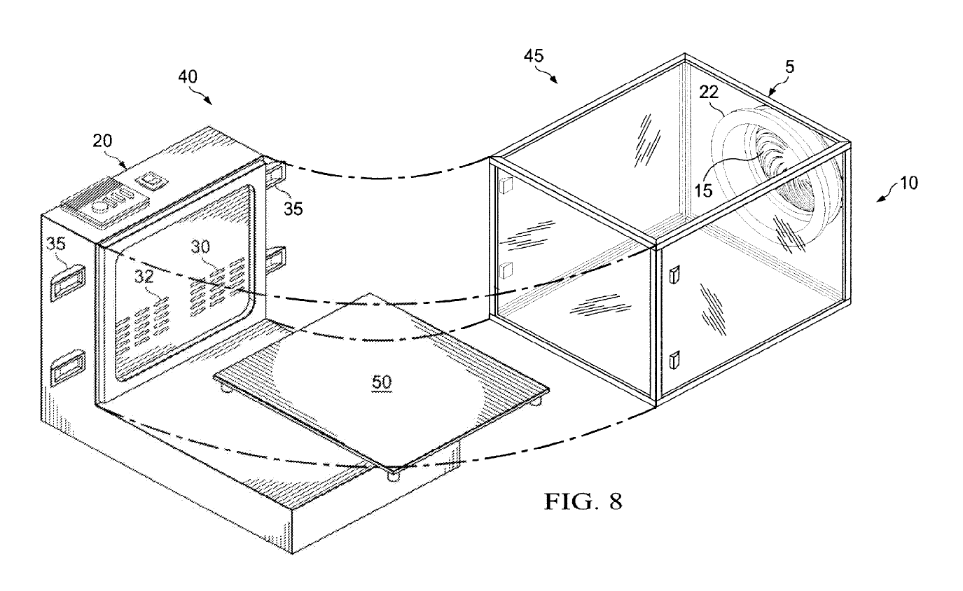

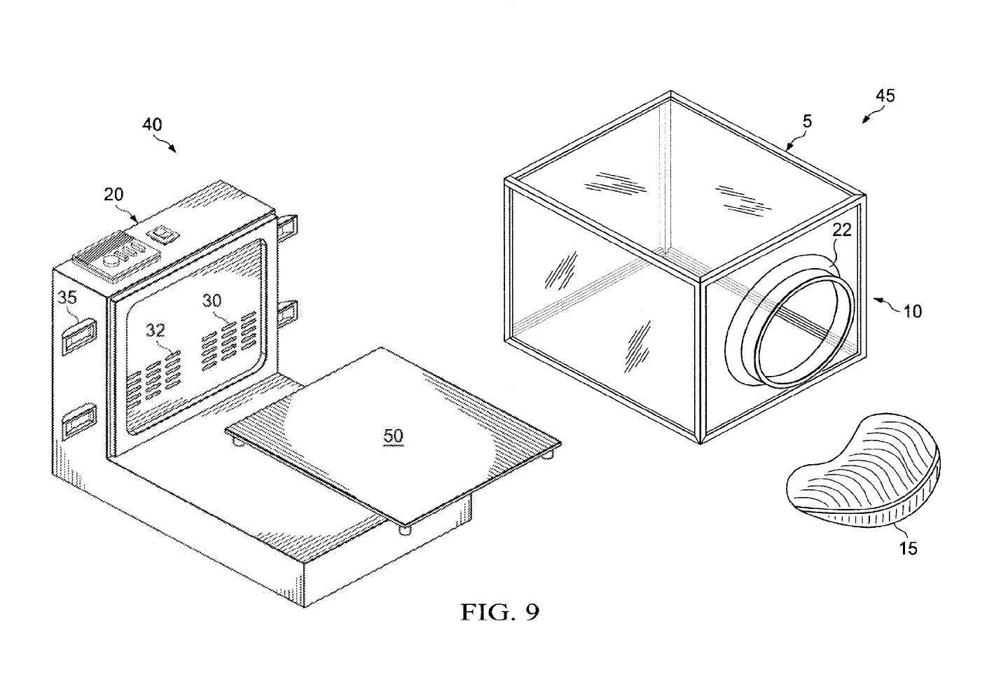

FIG. 8 and FIG. 9 show a complete apparatus for increasing venous blood flow in a preferred embodiment of the current invention. In this embodiment, a sterile plastic disposable tray 50 may be removed and cleaned or replaced, for example for each new user for sanitation reasons. The flexible iris 15, may also be removed and replaced for each new user.

In one embodiment, the invention may comprise a splash shield on the heated air vents 30, the cool air vents 32, the intake or the exhaust vents. This could act to prevent contamination or damage to the vent electrical components behind the vent as a result of splashing of fluids. In another embodiment, this could also act to prevent damage to the vent or electrical components behind the vent during cleaning. The splash guard may also comprise a HEPA filter or a replaceable filter.

The intake and exhaust vents may be positioned on the forced air unit 20 such that air can move from the heated air vents 30 into the detachable transparent housing 5. Exhaust vents may be positioned such that air can be vented out of the detachable transparent housing 5 as warm air is brought in through the intake vents.

In one embodiment, the power unit 40 detaches from the holding unit 45. The power unit 40 may be removably secured to the holding unit using clips 35, or another type of lock or fastener which may be conveniently operated by a user. This allows for the holding unit 45 to be cleaned or sterilized using methods which could be damaging to the power unit 40. In a preferred embodiment, the components of the power unit 40, or the holding unit 45, or both, are constructed using autoclavable materials, which may facilitate sterilizing the device after patient use. In a preferred embodiment, the holding unit 45 or power unit 40 further comprise a handle to facilitate moving the unit or both units to a convenient location. A preferred embodiment of the invention is a portable device, such that the device could be moved to a patient bedside for use. An embodiment of the invention is sufficiently lightweight that it could be carried by a user.

In one embodiment, the invention comprises a flexible iris 15 which is removable from the front portion 10 of the detachable transparent housing 5. The flexible iris may be disposable or autoclavable to facilitate maintenance of a sterile environment. In another embodiment, the invention comprises a detachable plastic tray which is disposable or autoclavable. In an embodiment, the detachable plastic tray may be ergonomically designed such that the wrist or arm of a user is oriented in a safe and comfortable position, or a position which facilitates a measurement to be taken from the patient.

In another embodiment, the environment inside the detachable transparent housing may be controlled by a thermostat, rheostat or the like. The power unit 40 may comprise a heating control 25. In some embodiments, the heating control may be recessed to prevent accidental adjustment of the control, or may comprise a code capable of locking the heating control to a certain temperature.

Further modification and alternative embodiments of this invention will be apparent to those skilled in the art in view of this description. Accordingly, this description is to be construed as illustrative only and is for the purpose of teaching those skilled in the art the manner of carrying out the invention. It is to be understood that the forms of the invention herein shown and described are to be taken as the presently preferred embodiments. Various changes may be made in the shape, size, and arrangement of pans. For example, equivalent elements or materials may be substituted for those illustrated and described herein, and certain features of the invention may be utilized independently of the use of other features, all as would be apparent to one skilled in the art after having the benefit of this description of the invention.

### REFERENCES CITED

The following references, to the extent that they provide exemplary procedural or other details supplementary to those set forth herein, are specifically incorporated herein by reference.

### U.S. Patent Documents

U.S. Pat. No. 7,014,431 issued on Mar. 21, 2006, with Hansen et al. listed as inventors.
U.S. Pat. No. 6,967,309 issued on Nov. 22, 2005, with Wyatt et al. listed as inventors.
U.S. Pat. No. 6,723,115 issued on Apr. 20, 2004, with Daly listed as the inventor.
U.S. Pat. No. 5,728,142 issued on Mar. 17, 1998, with Prokpchak et al. listed as inventors.

### U.S. Patent Publications

U.S. Patent Publication No. 2007/0068932 published on Mar. 29, 2007, with Hewes et al. listed as the inventors.

### Non-Patent Documents

Venodilation Techniques to Enhance Venepuncture and Intavenous Cannulation, Raymond J. Roberge, Journal of Emergency Medicine, Vol. 27, No. 1 pp. 69-73, 2004.
Local Warming and Insertion of Peripheral Venous Cannulas: Single Blinded Prospective Randomized Controlled Trial and Single Blinded Crossover Trial. Rainer Lenhardt, Tanja Seybold, Oliver Kimberger, Brigitte Stoiser and Daniel Sessler. British Medical Journal, Volume 325, 24 Aug. 2002.

What is claimed:

1. An apparatus for increasing venous blood flow of an extremity of a patient comprising:
a forced heated air unit having a heating element, a heating control, vents through which air can pass, and a fan to force heated air through the vents;
a detachable transparent housing having an interior adapted to receive said extremity in which said increased venous blood flow is desired, an exterior, and a substantially transparent portion;
an opening in the detachable transparent housing which permits introduction of said extremity into the detachable transparent housing; and
wherein the detachable transparent housing is capable of being fastened to the forced heated air unit such that air from the vents in the forced heated air unit can pass into the interior of the detachable transparent housing.

2. The apparatus of claim 1, further comprising a safety switch capable of shutting off the heating element if the temperature in the detachable transparent housing rises above a predetermined temperature.

3. The apparatus of claim 2, wherein the predetermined temperature is approximately 66° C.

4. The apparatus of claim 3, further comprising a fan within the detachable transparent housing capable of circulating air to achieve a uniform temperature within the detachable transparent housing.

5. The apparatus of claim 1, wherein the detachable transparent housing is capable of being removed for sterilization.

6. A method for increasing venous blood flow of an extremity of a patient, comprising the steps of:
a) providing the apparatus of claim 1;
b) applying power to the apparatus;
c) adjusting the heating control to achieve a predetermined temperature in the interior of the detachable transparent housing;
d) placing the extremity of the patient within the detachable transparent housing via the opening in the detachable transparent housing;
e) observing the extremity from the exterior of the apparatus until the venous blood flow increases.

7. The method of claim 6, wherein the predetermined temperature is less than approximately 66° C.

8. The method of claim 6, wherein the predetermined temperature is between approximately 55° C. and 61° C.

9. The method of claim 6, wherein the predetermined temperature causes the skin temperature of the extremity to be optimal for venepuncture.

10. The method of claim 6, wherein the predetermined temperature causes the skin temperature of the extremity to be between approximately 39° C. and 42° C.
